(19) United States
(12) Patent Application Publication
(10) Pub. No.: US 2025/0032258 A1
(43) Pub. Date: Jan. 30, 2025

(54) VALVE LEAFLET EXOSKELETON AND RELATED SYSTEMS AND METHODS

(71) Applicant: Kar Health, LLC, Irvine, CA (US)

(72) Inventors: Wassef Karrowni, Irvine, CA (US); Mirna Karouni, Irvine, CA (US)

(21) Appl. No.: 18/784,585

(22) Filed: Jul. 25, 2024

Related U.S. Application Data
(60) Provisional application No. 63/528,838, filed on Jul. 25, 2023.

(51) Int. Cl. A61F 2/24

(52) U.S. Cl. CPC A61F 2/2463; A61F 2/246; A61F 2210/0014; A61F 2220/0008; A61F 2220/0091

(57) ABSTRACT

A leaflet clip for repairing a valve, comprising a first arm having an adhering surface, a second arm, a hinge point connection the first arm and the second arm and forcing the first arm and second arm together, a delivery anchor disposed on the hinge point, a center plug extending from the hinge point, and at least one hook extending from the adhering surface. The leaflet clip configured for minimally invasive repair of a valve.

### CROSS-REFERENCE TO RELATED APPLICATION(S)

This application claims the benefit under 35 U.S.C. § 119 (e) to U.S. Provisional Application 63/528,838, filed Jul. 25, 2023, and entitled Valve Leaflet Exoskeleton, which is hereby incorporated herein by reference in its entirety for all purposes.

### TECHNICAL FIELD

The disclosure relates to various medical devices, systems and methods of use, and more particularly to devices, systems, and methods for enhancing and/or support the function of valve leaflets.

### BACKGROUND

As is understood, the human heart has four chambers and four valves, including the aortic valve. These valves open and shut to allow blood to pass from each chamber of the heart and prevent the backward flow of that blood. Similar valves are also found in the veins to aid to blood flow back to the heart.

As shown in FIG. 1, the aortic valve is typically composed of three leaflets. Each leaflet has a margin where it is attached to the aortic annulus in a semilunar fashion, and each leaflet has two free edges, each adjacent to and in contact with the two adjacent leaflets. As is understood, the free edges of each of the leaflets are thicker than the leaflet body. As a result of this anatomy, during valve closure the overlap of the free edges of adjacent leaflets serves to increase support of the valve. When the valve is closed, each of the three valve leaflets contain a sinus referred to, by those of skill in the art, as the sinus of Valsalva or aortic sinus. The sinuses of Valsalva are outpouching aortic wall structures that are demarcated by the insertion of each leaflet.

The body of each leaflet is thin and pliable but has a core of fibrous tissue with endothelial linings on its arterial and ventricular sides. During systole, or the phase of ventricular contraction of the cardiac cycle, the pressure in the left ventricle is increased and the aortic valve leaflets are pushed apart such that they fall back into their respective sinuses, allowing ejection of blood into the aortic root with no impediment on coronary blood flow.

During diastole, or the relaxation phase of the ventricle, the pressure in the ventricle drops below that in the aortic root and the aortic valve leaflets close, preventing regurgitation of the blood into the left ventricle. In a normal aortic valve, the adjacent leaflets coapt by at least 2 mm (coaptation height) to ensure valve competence (normal length of coaptations ranges from 2-6 mm). There are multiple other parameters that may be used to describe leaflet anatomy and function including geometric height, effective height, and commissure height, as would be understood by those of skill in the art.

The normal anatomy of the aortic valve is tricuspid, meaning it has three leaflets, but a common cardiac valvular anomaly is a bicuspid aortic valve (with two leaflets), occurring in 1-2% of the general population. A bicuspid aortic valve results from fusion of aortic valve leaflets and occurs most commonly #4057966 v.2 1(approximately 80%) between the right coronary and left coronary leaflets with secondary association with future complications such as insufficiency and/or stenosis.

It would be understood that there are two typical forms of valve malfunction (which can exhibit themselves separately or in combination in patients): (1) insufficiency (leakage of the valve) and (2) stenosis (narrowing of the valve).

Aortic valve insufficiency (AI) or regurgitation is a condition in which the aortic valve does not close properly, leading to blood flowing backwards in the heart (back into the left ventricle) during diastole instead of pumping out to the body organs. With AI the heart compensates by pumping harder, which, over time, can lead to weakening of the heart muscle and ultimately heart failure.

The prevalence of AI increases with advancing age, and the prevalence of moderate or severe AI has been estimated to be 1.6% of individuals≥65 years. AI can result from different pathophysiologic mechanisms, including a dilated aortic annulus, single leaflet prolapse in tricuspid aortic valves, conjoined leaflet prolapse in bicuspid aortic valves, or perforation/tear in the leaflet body. The dilated aortic annulus results in a sagging of the belly of the leaflets resulting in lack of central leaflet apposition.

Conventional treatment for insufficiency and stenosis is surgery with replacement of the aortic valve using either a prosthetic mechanical valve or prosthetic tissue valve. Disadvantages of aortic valve replacement include possible late prosthetic valve dysfunction secondary to structural failure, bacterial infection of the prosthesis, and/or the need to keep the patient on blood thinners.

One solution for these limitations has been the development of surgical aortic valve repair methods to restore normal function of the aortic valve, instead of replacement. Such known methods have exhibited safe and excellent immediate results with encouraging durability with up to 15-20 years of follow-up. These methods are highly invasive with many of the complications of invasive procedures.

Transcatheter aortic valve implantation (TAVI) is a less invasive procedure that has been introduced to treat the narrowing of the aortic valve (aortic valve stenosis). TAVI has proved to be as effective, and safer, than the traditional surgical replacement.

However, in the case of pure native AI, TAVI is not considered a safe alternative for surgical replacement or repair because of the absence of significant leaflet or annular calcifications in most cases of pure AI, calcification being required to anchoring. The positioning of the prosthesis in TAVI in the correct position depends on anchoring on the calcifications found in cases of aortic valve stenosis. Thus, TAVI for patients with pure AI carries certain potential risks including malpositioning due to inadequate sealing, valve embolization, and significant leaking around the valve (paravalular regurgitation). Oversizing of the TAVI prosthesis in an attempt to better anchor and seal the device also involves a risk of valve dislocation, conduction disorders, and annulus rupture.

To date, there are no non-invasive or minimally invasive methods or systems to repair the aortic valve in cases of AI. Even though standard surgical repair of the valve is an established effective method, it is a very invasive method with several risks including death, stroke, bleeding, infection, heart rhythm problems, blood clots, significant discomfort, an extended hospital stay, and prolonged recovery, among others. Further, it has been estimated that 7.8% of patients with severe AI, who needed a surgical intervention, did not get treated because of advanced age and multiple comorbidities making surgery an excessive risk.

There is a need in the art for an improved device and related non-invasive or less-invasive methods for aortic valve repair in the treatment of AI and related valvular repairs.

### BRIEF SUMMARY

Disclosed herein are various a valve enhancement or support devices. Particularly disclosed is a clip for implantation into a valve by positioning the clip over a free edge of a valve leaflet, such as the aortic valve or a venous valve, in order to enhance or support deficient leaflet tissue, and related methods and systems. The various devices and methods disclosed herein are configured to support native leaflet tissue that is deficient for one or more reasons, including prolapse, perforation, and/or restricted mobility, thereby treating insufficient valvular function.

In Example 1, a valve clip, comprising a first arm and a second arm attached at a hinge point and an adhering surface at an end of the first arm, wherein the hinge is configured for placement at a free edge of a valve leaflet and the first arm and second arm are on opposing sides of the valve leaflet.

Example 2, related to the valve clip of any of Examples 1 and 3-8, further comprising an anchor at the hinge point configured for attaching the valve clip to a delivery device.

Example 3, related to the valve clip of any of Examples 1-2 and 4-8, further comprising one of more hooks on a surface of the adhering surface.

Example 4 related to the valve clip of any of Examples 1-3 and 5-8, wherein the adhering surface comprises a membrane.

Example 5 related to the valve clip of any of Examples 1-4 and 6-8, wherein the first arm, second arm, and adhering surface are shaped from a frame comprising nitinol.

Example 6 related to the valve clip of any of Examples 1-5 and 7-8, wherein the adhering surface is shaped to cover substantially all of the valve leaflet.

Example 7 related to the valve clip of any of Examples 6 and 8, further comprising a center plug extending from the hinge point.

Example 8 relates to a method for repairing a valve comprising inserting via minimally invasive techniques valve clip of any of Examples 1-7.

In Example 9, a clip for placement on a valve leaflet comprising a leaflet portion configured for securement against a first side of the valve leaflet, a rear portion configured for securement against a second side of the valve leaflet, and a hinge point connecting the leaflet portion and the rear portion.

Example 10 relates to the clip of any of Examples 9 and 11-19, wherein the rear portion comprises a rim extending along substantially all of the leaflet portion.

Example 11 relates to the clip of any of Examples 9-10 and 12-19, wherein the rear portion comprises a split rim.

Example 12 relates to the clip of any of Examples 9-11 and 13-19, wherein the rear portion comprises a flared extension extending from the center of the leaflet portion.

Example 13 relates to the clip of any of Examples 9-12 and 14-19, wherein the leaflet portion is shaped to conform with leaflet anatomy and cover substantially all of the leaflet.

Example 14 relates to the clip of any of Examples 9-13 and 15-19, further comprising an anchor point on the hinge point.

Example 15 relates to the clip of any of Examples 9-14 and 16-19, further comprising a center plug extending from the hinge point.

Example 16 relates to the clip of any of Examples 9-15 and 17-19, further comprising at least one hook extending from the leaflet portion configured for attachment to the leaflet.

Example 17 relates to the clip of any of Examples 9-16 and 18-19, wherein the leaflet portion comprises a spring coil frame.

Example 18 relates to the clip of any of Examples 9-17 and 19, wherein the leaflet portion comprises a membrane.

Example 19 relates to a method for repairing a valve leaflet, comprising inserting the device of any of Example 9-18 into a valve.

In Example 20, a leaflet clip for repairing a valve, comprising a first arm having an adhering surface, a second arm, a hinge point connection the first arm and the second arm and forcing the first arm and second arm together, a delivery anchor disposed on the hinge point, a center plug extending from the hinge point, and at least one hook extending from the adhering surface.

In any of the Examples, the frame of the clip wraps around the free edge of the leaflet and is anchored either passively or actively. Passive anchoring is due to characteristics of the material of the device and active anchoring includes using one of the arms as the anchor or other mechanism.

In any of the Examples, the adhering surface of the arm is shaped to support the tissue in a prolapsed leaflet, in effort to restore normal leaflet shape.

In any of the Examples, the upper edge (hinge point) of the device provides a supportive surface that seals the gap with the adjacent leaflets when the valve is closed.

In any of the Examples, the edges of the device are foldable to assist with delivery of the device.

In any of the Examples, the defective leaflet is covered or supported by multiple complementary devices that are deployed adjacent or overlapping to each other.

In any of the Examples, the adhering surface/arm is made of wire mesh inside which is embedded a membrane, or a membrane or fabric of material supported by metallic wires embedded in the membrane.

In any of the Examples, the device is used to correct a defective leaflet that may have a perforation, prolapse, or be retracted.

In any of the Example, the 3D dimensions of the adhering surfaces and their continuation may be specific for each individual leaflet based on preimplantation anatomic assessment for example using Computed Tomography (CT) scan imaging.

In any of the Example, the membrane enhances the free edge of the defective leaflet or extends it to improve approximation with adjacent leaflets in cases of prolapse or retraction.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a perspective view of an aortic valve with a perforation/defect, according to one implementation.

FIG. 2 is a perspective view of a leaflet clip, according to one implementation

FIG. 3 is a side view of a valve having a leaflet clip in place, according to one implementation.

FIG. 4 is a side view of a valve having a leaflet clip in place, according to one implementation.

FIG. 5 is a side view of a valve having a leaflet clip in place with a center plug, according to one implementation.

FIG. 6 is a side view of a valve having a leaflet clip in place with a center plug, according to one implementation.

FIG. 7 is a top view of a valve having a leaflet clip in place, according to one implementation.

FIG. 8 is a top view of a valve having a leaflet clip in place with spring coils, according to one implementation.

FIG. 9 is a top view of a valve having a leaflet clip in place with spring coils, according to one implementation.

FIG. 10 is a top view of a valve having a leaflet clip in place, according to one implementation.

FIG. 11 is a top view of a valve having a leaflet clip in place with a center plug, according to one implementation.

FIG. 12 is a top view of a valve having a leaflet clip in place with a center plug, according to one implementation.

FIG. 13 is a perspective view of a leaflet clip having a wide, short rim, according to one implementation.

FIG. 14A is a rear view of a leaflet clip having a wide, tall rim in place on a leaflet, according to one implementation.

FIG. 14B is a perspective view of a leaflet clip having a wide, tall rim in place on a leaflet, according to one implementation.

FIG. 14C is a cross-section view of a leaflet clip having a wide, tall rim in place on a leaflet, according to one implementation.

FIG. 14D is a perspective view of a leaflet clip having a wide, tall rim, according to one implementation.

FIG. 15A is a top, perspective view of a leaflet clip having a split, short rim in place on a leaflet, according to one implementation.

FIG. 15B is a rear view of a leaflet clip having a split, short rim, according to one implementation.

FIG. 15C is a cross-section view of a leaflet clip having a split, short rim, according to one implementation.

FIG. 16A is a rear, perspective view of a leaflet clip having a split, tall rim, according to one implementation.

FIG. 16B is a perspective view of a leaflet clip having a split, tall rim in place on a leaflet, according to one implementation.

FIG. 17A is a rear view of a leaflet clip having a narrow, tall rim in place on a leaflet, according to one implementation.

FIG. 17B is a rear view of a leaflet clip having a narrow, tall rim, according to one implementation.

FIG. 17C is a cross-section view of a leaflet clip having a narrow, tall rim, according to one implementation.

FIG. 17D is a cross-section view of a leaflet clip having a narrow, tall rim in place on a leaflet, according to one implementation.

FIG. 18A is a rear view of a leaflet clip having a tall, shaped rim extension, according to one implementation.

FIG. 18B is a cross-section view of a leaflet clip having a tall, shaped rim extension, according to one implementation.

FIG. 18C is a rear view of a leaflet clip having a tall, shaped rim extension in place on a leaflet, according to one implementation.

FIG. 18D is a cross-section view of a leaflet clip having a tall, shaped rim extension in place on a leaflet, according to one implementation.

FIG. 18E is a front view of a leaflet clip having a tall, shaped rim extension, according to one implementation.

FIG. 18F is a cross-section view of a leaflet clip having a tall, shaped rim extension in place in a valve, according to one implementation.

FIG. 18G is a top, perspective view of a leaflet clip having a tall, shaped rim extension in place in a valve, according to one implementation.

FIG. 18H is a top, perspective view of a leaflet clip having a tall, shaped rim extension in place in a valve, according to one implementation.

FIG. 18I is a center, cross-section view of a valve having a leaflet clip having a tall, shaped rim extension in place, according to one implementation.

FIG. 18J is a side, cross-section view of a valve having a leaflet clip having a tall, shaped rim extension in place, according to one implementation.

FIG. 18K is a side, cross-section view of a valve having a leaflet clip having a tall, shaped rim extension in place, according to one implementation.

FIG. 19 is a rear perspective view of a leaflet clip having an anchor in place on a leaflet, according to one implementation.

FIG. 20 is a side, perspective view of a leaflet clip having an anchor, according to one implementation.

FIG. 21 is a rear, perspective view of a leaflet clip having an anchor, according to one implementation.

FIG. 22 is a top, perspective view of a leaflet clip having an anchor in place in a valve, according to one implementation.

FIG. 23 is a bottom, perspective view of a leaflet clip having a center plug in place in a valve, according to one implementation.

FIG. 24 is a perspective view of a leaflet clip having a center plug, according to one implementation.

FIG. 25 is a perspective view of a leaflet clip having a center plug in place in a valve, according to one implementation.

FIG. 26 is a perspective view of a multi-component leaflet clip, according to one implementation.

FIG. 27 is a perspective view of a multi-component leaflet clip in place on a leaflet, according to one implementation.

FIG. 28 is a perspective view of a multi-component leaflet clip in place in a valve, according to one implementation.

FIG. 29 is a perspective view of a multi-component leaflet clip in place on a leaflet, according to one implementation.

FIG. 30 is a rear, perspective view of a multi-component leaflet clip in place on a leaflet, according to one implementation.

FIG. 31 is a perspective view of a multi-component leaflet clip in place in a valve, according to one implementation.

FIG. 32A is a rear view of a leaflet clip having attachment hooks, according to one implementation.

FIG. 32B is a rear, perspective view of a leaflet clip having attachment hooks, according to one implementation.

FIG. 32C is a side, perspective view of a leaflet clip having attachment hooks, according to one implementation.

FIG. 32D is a close-up view of a leaflet clip having attachment hooks, according to one implementation.

FIG. 33A is a rear view of a leaflet clip having attachment hooks in place in a valve, according to one implementation.

FIG. 33B is a cross-section view of a leaflet clip having attachment hooks in place in a valve, according to one implementation.

FIG. 34A is a close-up, cross-section view of a leaflet clip having attachment hooks in place in a valve, according to one implementation.

FIG. 34B is a close-up, cross-section view of a leaflet clip having attachment hooks in place in a valve, according to one implementation.

FIG. 35A is a rear view of a leaflet clip having attachment hooks in place on a leaflet, according to one implementation.

FIG. 35B is a close-up view of a leaflet clip having attachment hooks in place on a leaflet, according to one implementation.

FIG. 36A is a side, cross-section view of a valve having a leaflet clip having attachment hooks in place, according to one implementation.

FIG. 36B is a cross-section view of a valve having a leaflet clip having attachment hooks in place, according to one implementation.

FIG. 37 is a rear view of a leaflet clip in place on a valve leaflet with a perforation, according to one implementation.

FIG. 38 is a top perspective view of a leaflet clip in place on a valve, according to one implementation.

### DETAILED DESCRIPTION

The various implementations herein relate to one or more devices for use in enhancing/supporting valve function. Particularly, the devices are configured to enhance/support leaflet function of a valve, such as the aortic valve or a venous valve, to address valvular malfunction. The devices may be used in connection with a variety of valves including, for example, the aortic valve.

These various device implementations may serve as a repair mechanism for a leaky valve, as a result of valve insufficiency or regurgitation. In alternative implementations, the device may be used as an anchor for future or simultaneous transcutaneous valve implantation.

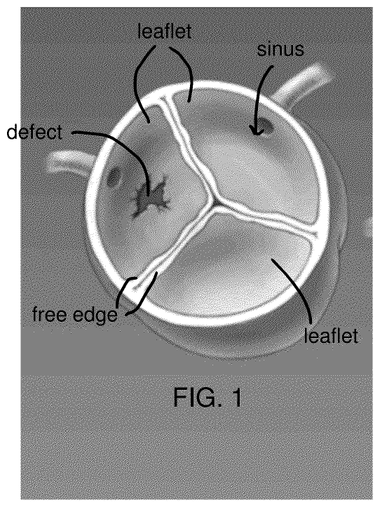

The various implementations described herein provide minimally/less invasive methods and systems for repair of valves and treatment of valvular insufficiency and/or regurgitation. In certain implementations, the various clip implementations described herein include a membrane to seal a perforation (defect) in the valve leaflet, such as that shown in FIG. 1, or to support prolapsing tissue. The procedures performed using the various clip implementations herein may be described as a percutaneous patch repair of an insufficient valve leaflet.

In various further implementations, the clip/device is a supporting frame or skeleton to restore the shape of a prolapsing leaflet tissue. Still further, the various device implementations described herein can be used as a spacer in order to fill a residual gap in the middle of the valve between the adjacent leaflets. These adjustments to the valve anatomy may restore the coaptation of the leaflets of the valve, thereby decreasing regurgitation. Further, the various clip implementations can also be used as anchors in order to stabilize subsequent delivery of transcatheter aortic valve implantation (TAVI), as noted above.

According to certain implementations, the disclosed clips can be implanted in a minimally invasive manner using a catheter, guide wire, or the like, as would be understood. In use according to certain implementations, the device is implanted into the free edge of valve leaflet by positioning the device in the center portion of the valve, as will be discussed further below. In various cases, one or more clips are used, with each clip being positioned over a different leaflet of the valve. That is, the treatment of a valve may include one device for each leaflet, more than one device for a single leaflet, and/or one or more devices for additional leaflets. In cases where more than one leaflet is treated, the space-filling material or the spacers on the multiple devices may work together in order to achieve the mission of sealing any gap between the leaflets during diastole, as will be discussed further below. Various additional or alternative procedures and treatments are possible using the described devices, as would be understood by those of skill in the art.

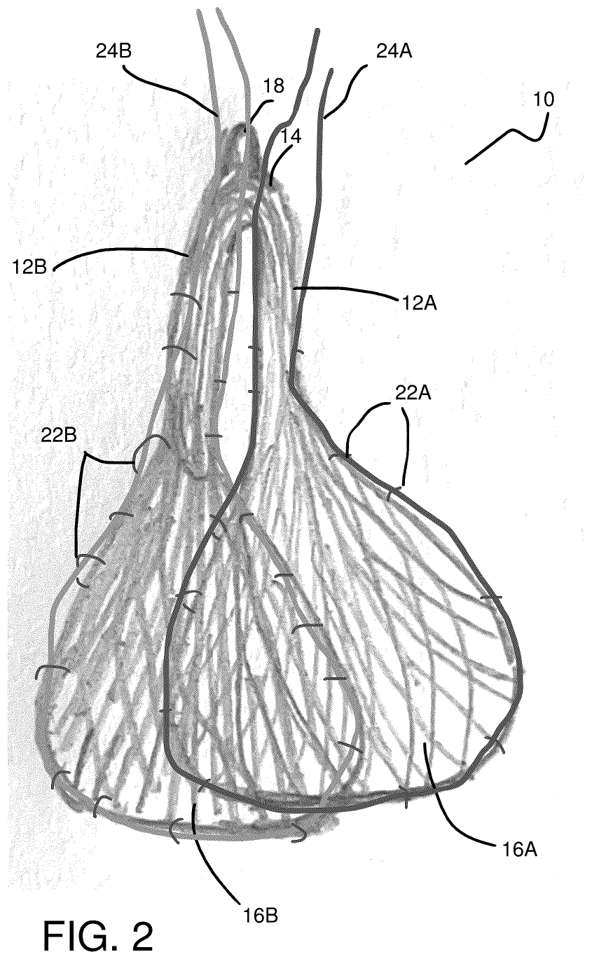

Turning to the figures in further detail, FIG. 2 shows one exemplary implementation of the device 10. In various implementations, the clip 10 includes two arms 12A, 12B attached at a central hinge point 14. In certain implementations, the clip 10 includes an adhering surface 16A, 16B (also referred to as a “surface” “body” or “leaflet portion”) at the distal end of each arm 12A, 12B. In certain implementation, the adhering surface 16A, 16B extends directly from the hinge point 14, as will be discussed further herein.

In various implementations, the hinge point 14 is configured to force the arms 12A, 12B together and hold the adhering surfaces 16A, 16B in contact with the leaflet 2 tissue, when the clip 10 is in place in a valve. That is, the clip 10 may be opened (flexed apart at the hinge point 14) for placement on the leaflet 2, where the leaflet tissue is sandwiched between portion of the clip 10, and then the clip 10 may passively or actively close at the hinge point 14 to adhere/affix/attached to the leaflet 2. In various implementations, the hinge point 14 is a self-closing passive mechanism or alternatively is an active forced mechanism, such as a spring, loaded hinge, screw-in mechanism, or similar active mechanism as would be understood. In implementations where the hinge point 14 is self-closing the device 10 may be made from a memory material where when forced out of shape the material returns to its original shape with the force is removed.

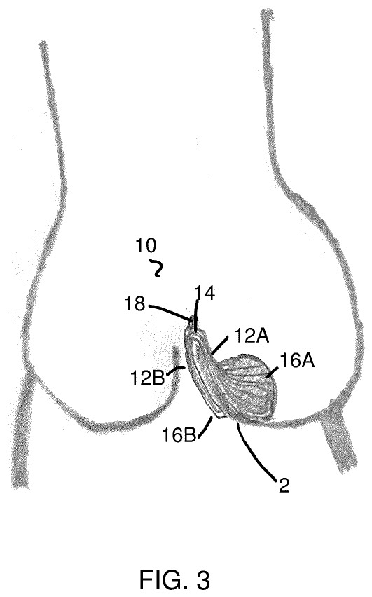

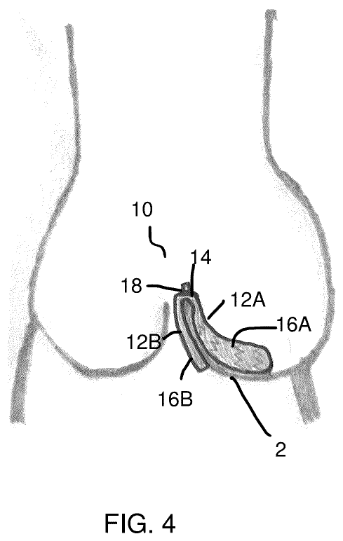

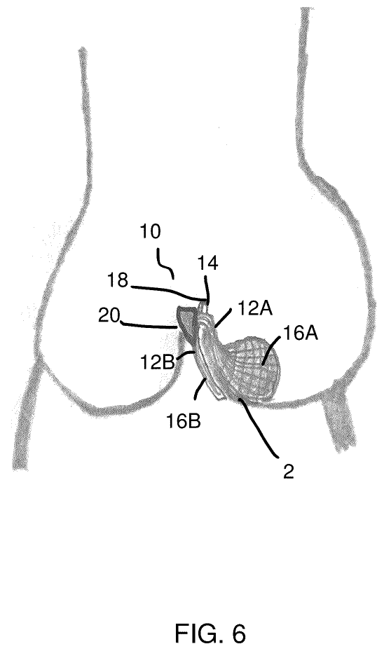

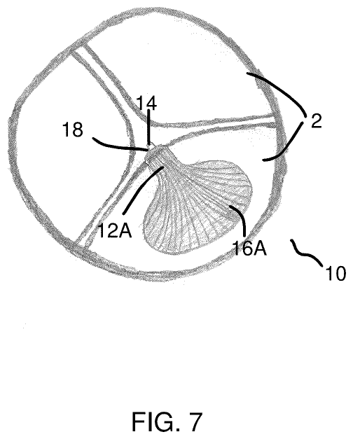

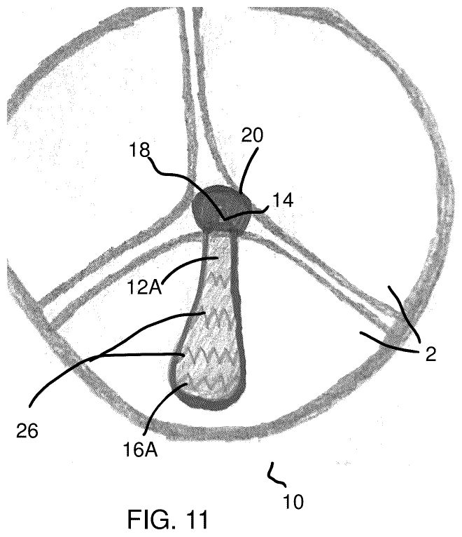

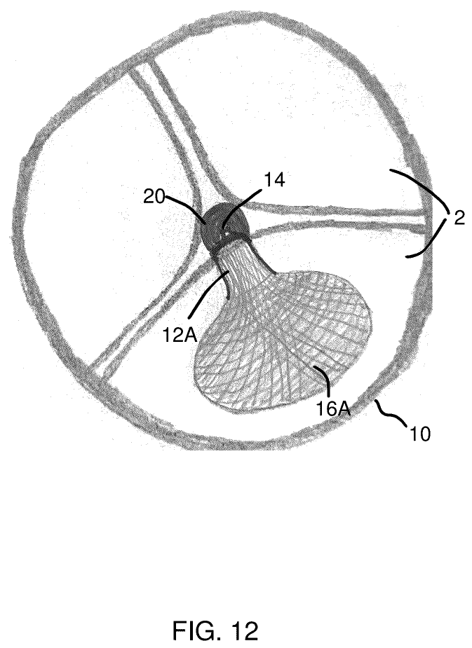

In various implementations, the clip 10 is configured to attach to one leaflet 2 where the leaflet tissue is sandwiched between the arms 12A, 12B, as shown in FIGS. 3-6. In various implementations, the clip 10 may be positioned in the middle of the valve leaflet 2 free edge, as shown in FIGS. 7-12, various alternative positions are possible. Turning back to FIGS. 3-6, as can be seen, one arm 12B of the clip 10 is positioned on the ventricular side of the aortic valve leaflet 2 while the other arm 12A is positioned on the aortic side of the aortic valve leaflet 2.

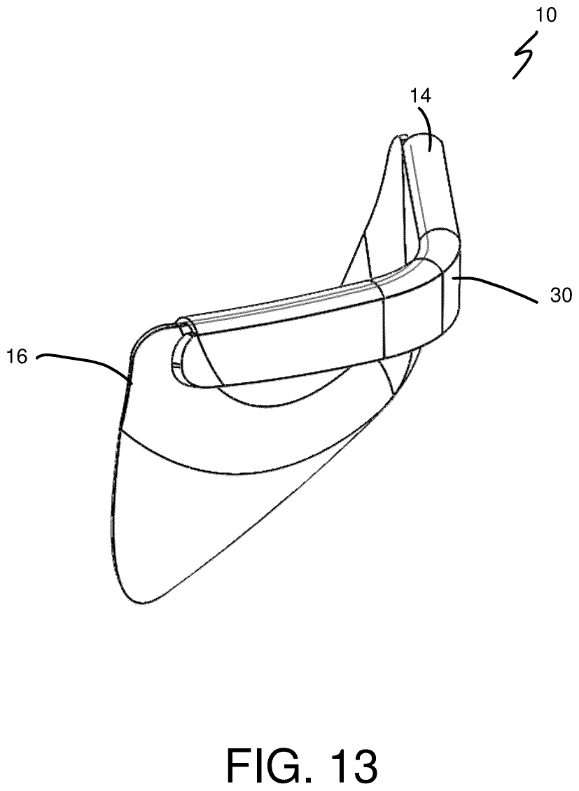

In various implementations, the arms 12A, 12B and adhering surfaces 16A, 16B of the clip 10 have a symmetric shape, as shown in FIG. 2, where both sides of the clip 10 have the same shape. In alternative implementations, the arms 12A, 12B and/or adhering surfaces 16A, 16B are asymmetric, having different shapes, such as shown in FIGS. 13-19. In various implementations, the side (adhering surface 16A) of the clip 10 placed facing the aorta is shaped and optionally configured to be a sealing membrane or patch, as will be discussed further below.

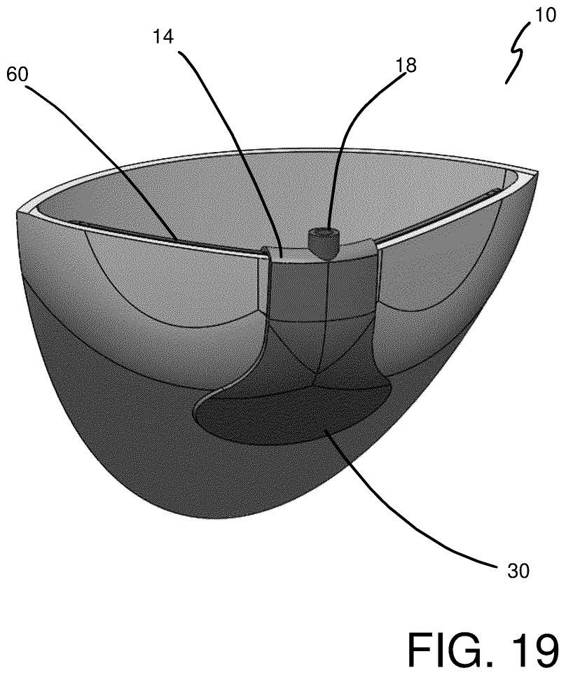

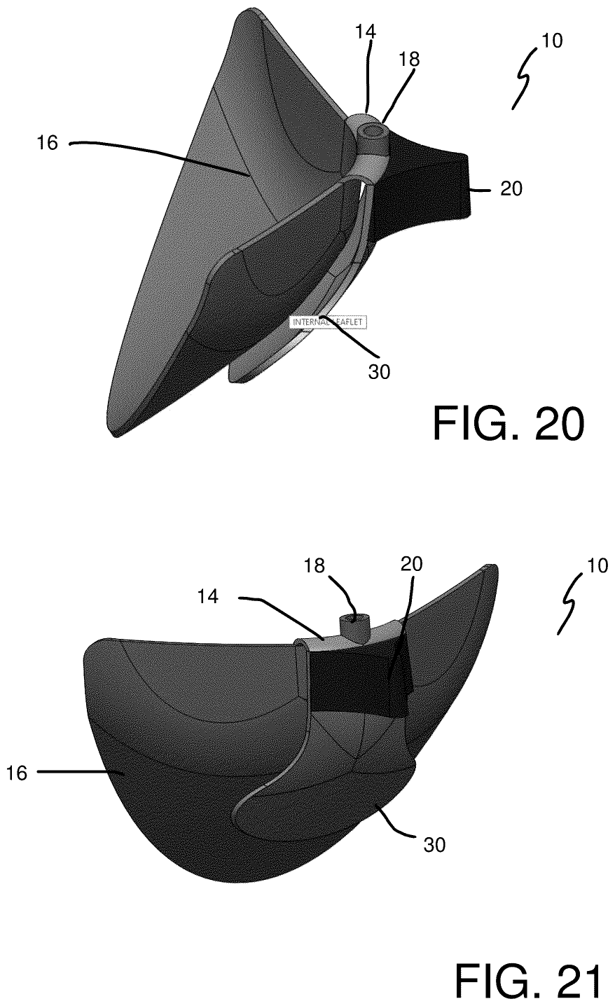

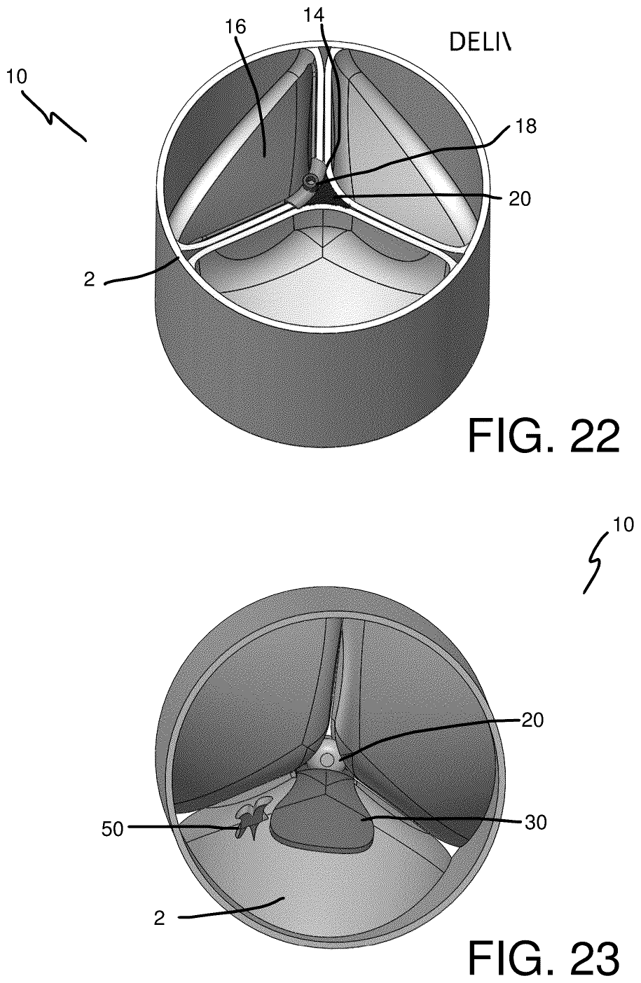

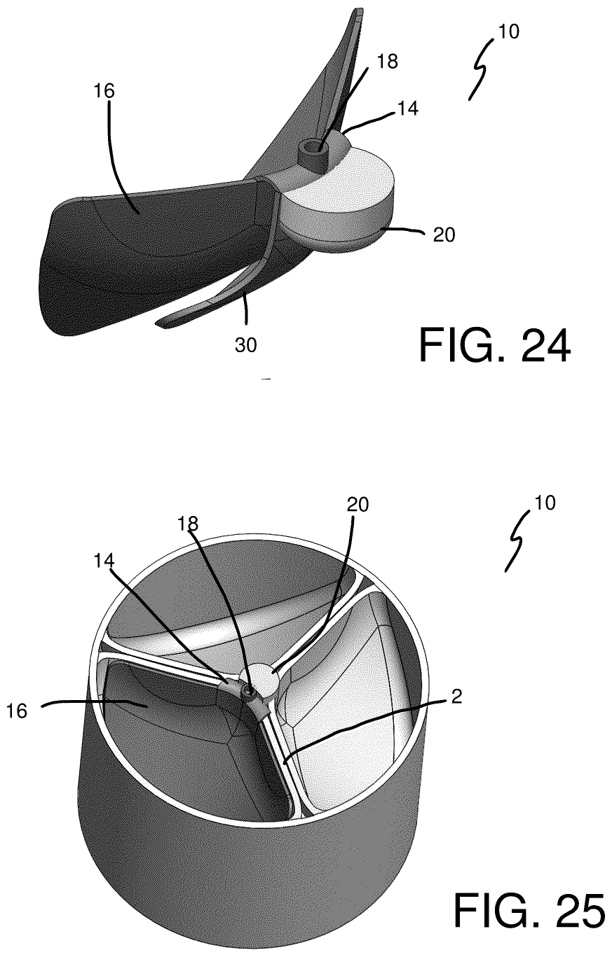

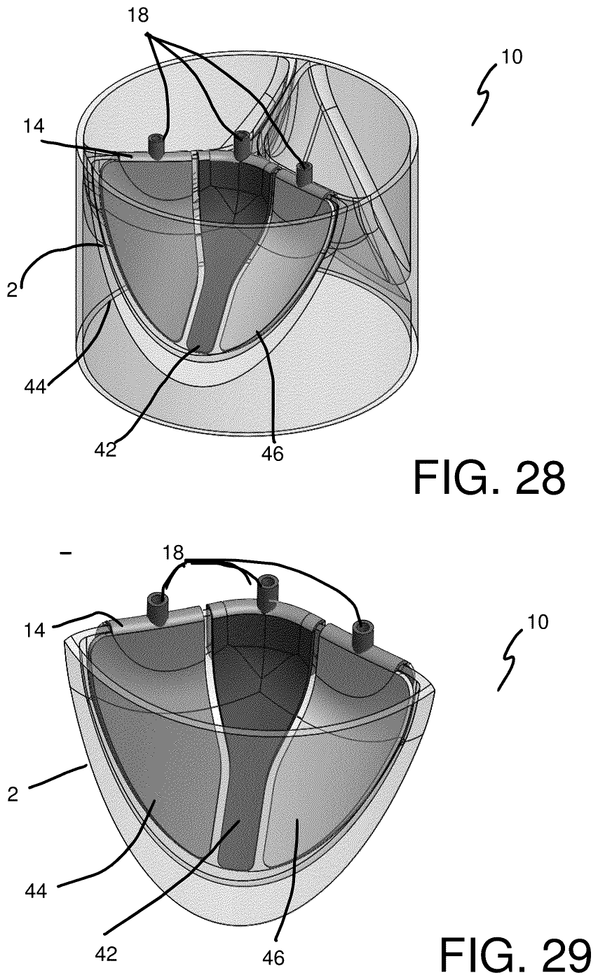

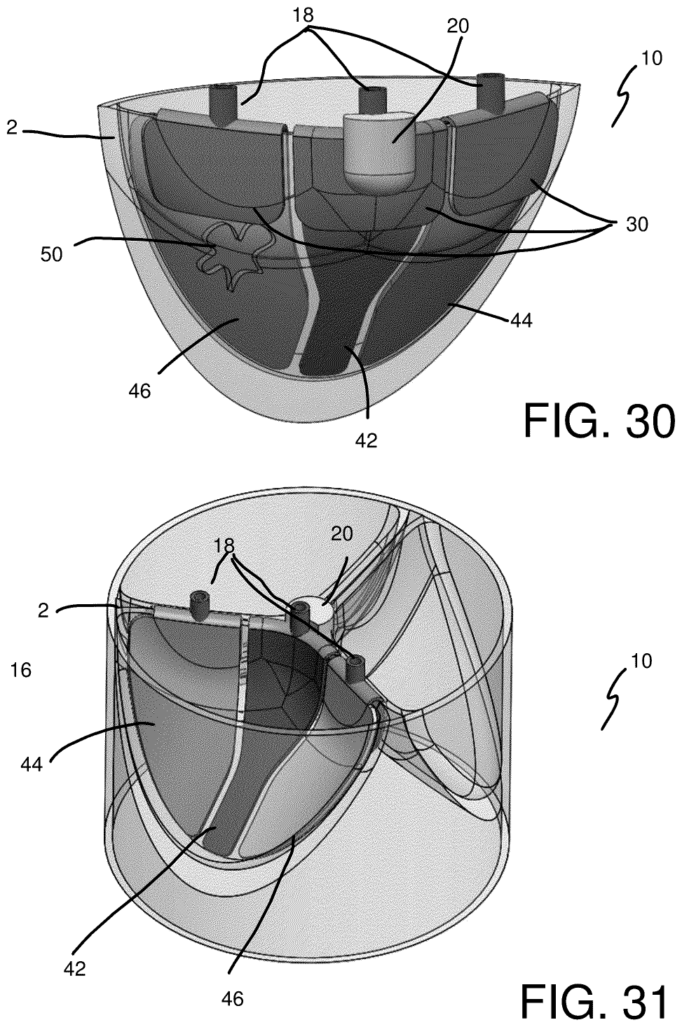

In various implementations, the device 10 may include an anchor/delivery feature 18, discussed further herein and in relation to FIGS. 19-23. Certain implementations may also include a central plug/extension, discussed further in relation to FIGS. 24-25. In certain implementation, the device 10 may have several separate components, as will be discussed further below in relation to FIGS. 28-31. The device 10 may include various combinations of features discussed herein.

Additionally, in some implementations, the arms 12A, 12B and/or adhering surfaces 16A, 16B may be of same length or different length depending on the correction/procedure to be completed. In certain implementations, the device 10 includes one arm 12A and adhering surface 16A and a rim or other feature to hold the clip 10 in place on the leaflet.

In certain implementations, one arm 12A, 12B and corresponding adhering surface 16A, 16B is a rod, optionally of wire mesh.

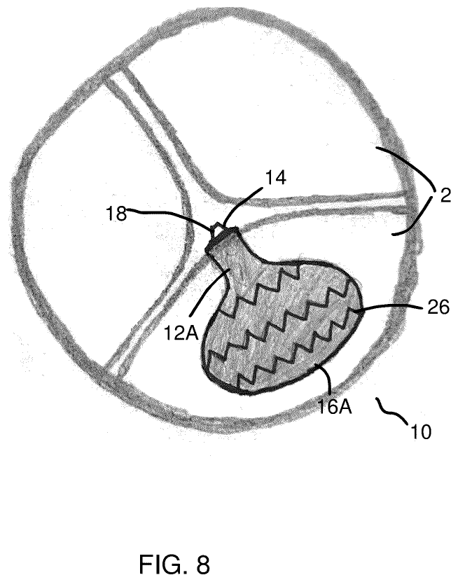

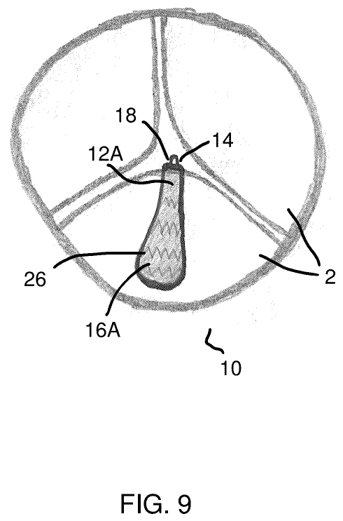

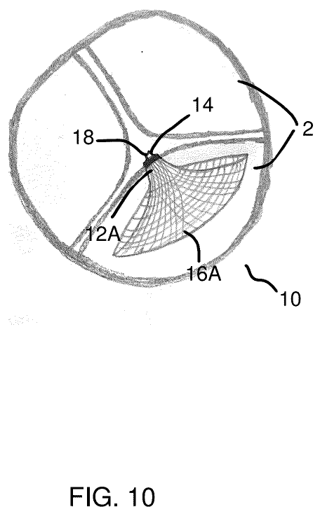

Continuing with FIG. 2, in various implementations, the clip 10 adhering surface 16A, 16B is a sealing membrane or patch or is partially made of a sealing membrane or patch. The sealing membrane or patch may have various dimensions and shapes depending on the procedure or defect to be cured. In various implementations the adhering surface 16A, 16B includes membrane tissues supported by a body shaft or skeleton composed of wires that are in continuity with the arm 12A, 12B of the clip 10 (shown for example in FIGS. 8 and 9). In these and other implementations, the wires could be sewed on the membrane tissue or alternatively the wires could form a two-layered mesh around the membrane tissue (shown for example in FIG. 10).

In various implementations, the clip 10 is made of one continuous metal frame, where the metal frame forms an outline of each adhering surface and arm, shown in FIG. 2. In certain implementations, the interior of the frame includes a mesh of further metal or other material. In certain implementations, the interior of the frame also includes a membrane.

In certain implementations, the frame for the membrane is composed of parallel lines of spring coil 26 (shown for example in FIGS. 8 and 9).

The composition of the membrane tissue could be pericardial material, polytetrafluoroethylene (PTFE), or other similar compatible material, as would be understood. The shape of the membrane, including length, and width could vary in order to cover various proportions of the leaflet 2 surface. The curvature of the membrane surface could also vary depending on the specific anatomy of the leaflet 2 to be treated.

The device 10, including the arms 12A, 12B and optionally the adhering surfaces 16A, 16B, may be made of any known shape-memory material. For example, in certain implementations, the device 10, arms 12A, 12B, adhering surface 16A, 16B can be made of nitinol. Alternatively, the device 10/arms 12A, 12B can be made of another metal such as Elgiloy, Phynox, titanium, a titanium alloy, or a stainless steel alloy. In a further alternative, any known shape-memory metal or other material can be used, as would be understood by those of skill in the art.

In various implementations, the arms 12A, 12B, and alternatively the entire clip 10, can be covered in a polymeric material such as PTFE. Further any portion or selection of portions of the clip 10 may be covered in a polymeric material. Alternatively, the material can be any known material that promotes the endothelization and covering of the device 10 with body tissue.

The arms 12A, 12B may optionally be plates or wires of varying width or diameter. Alternatively, the arms 12A, 12B can have any known shape or configuration. In addition, the arms 12A, 12B can vary in length depending on the anatomical and functional characteristics of the target valve.

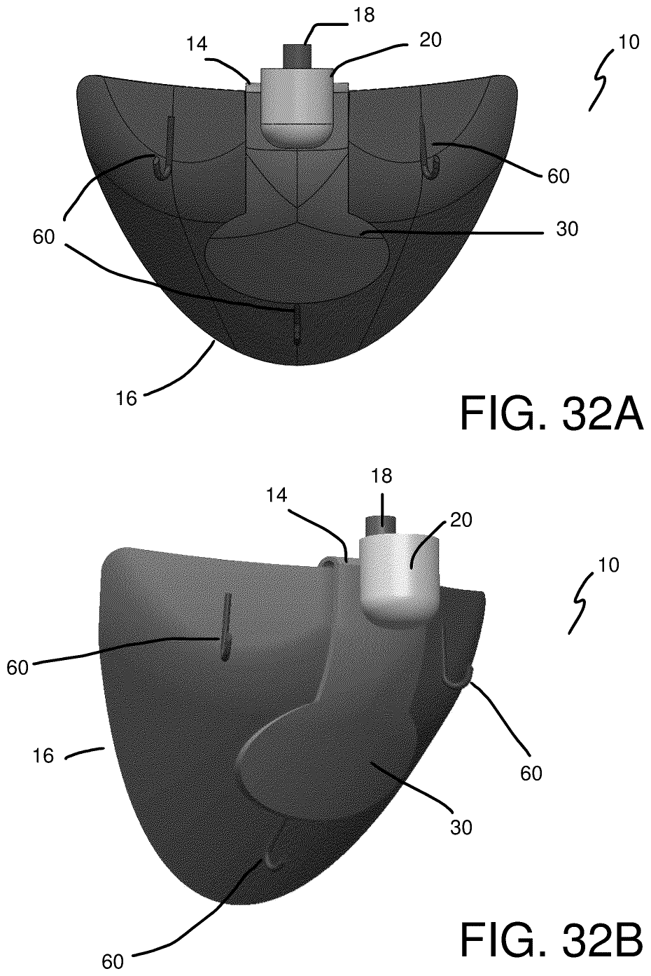

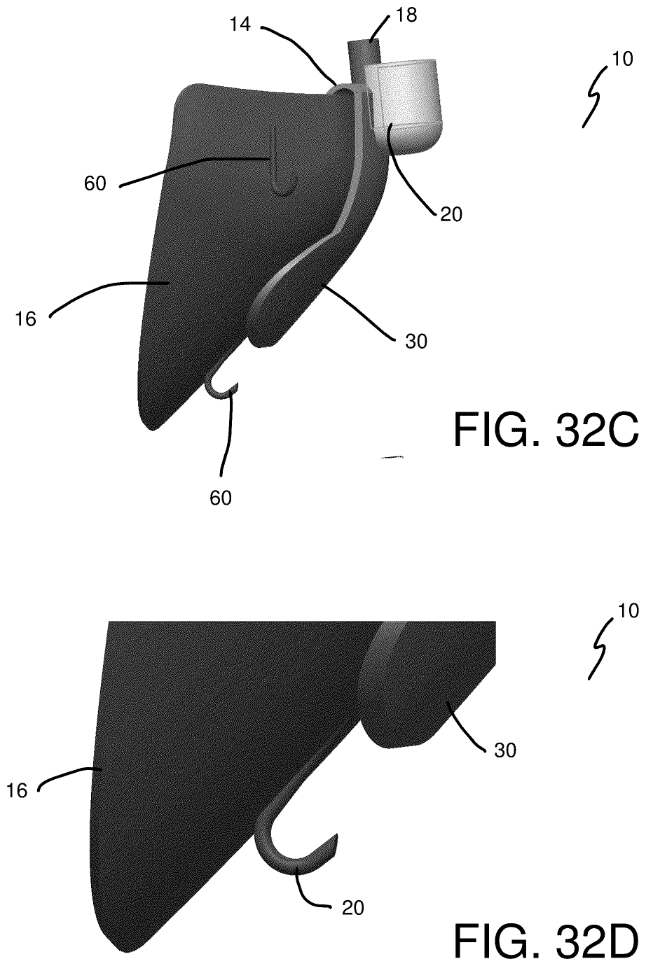

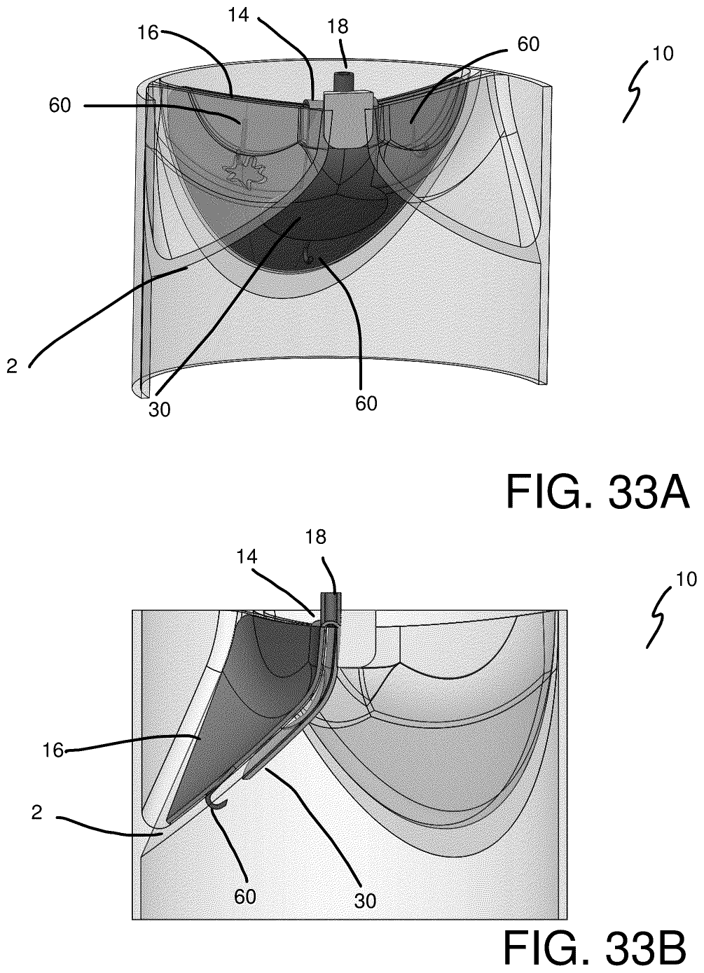

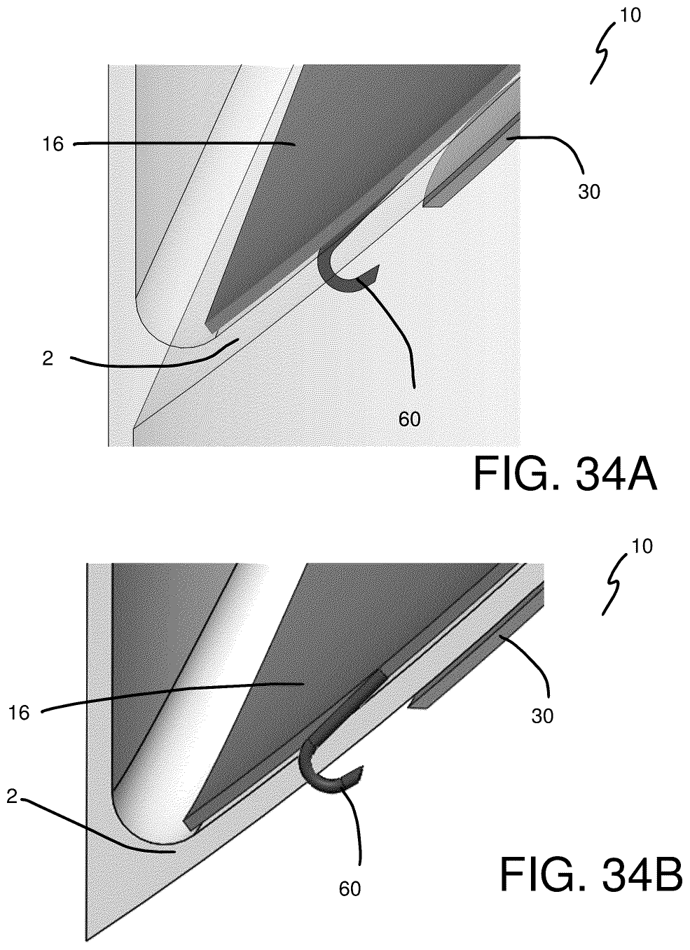

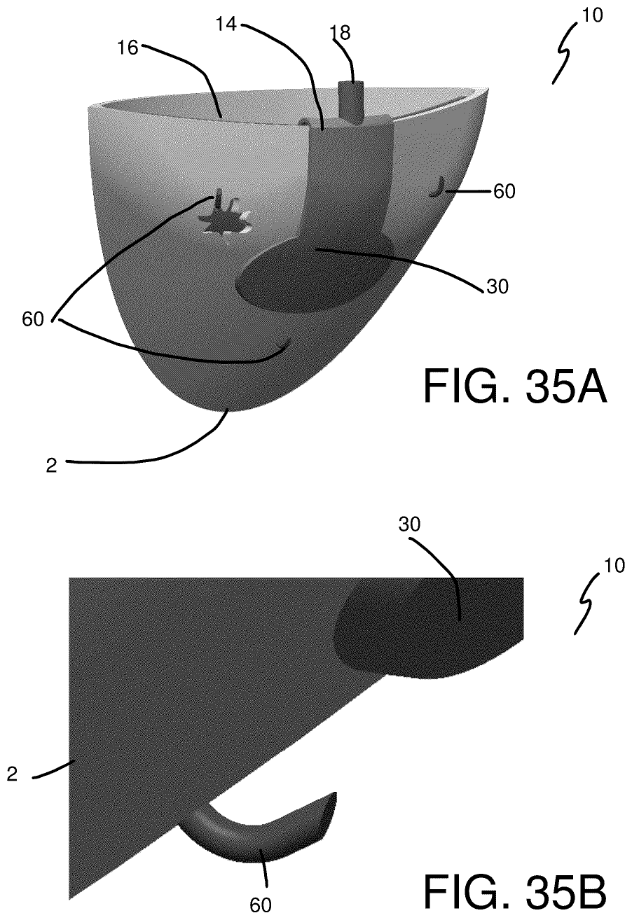

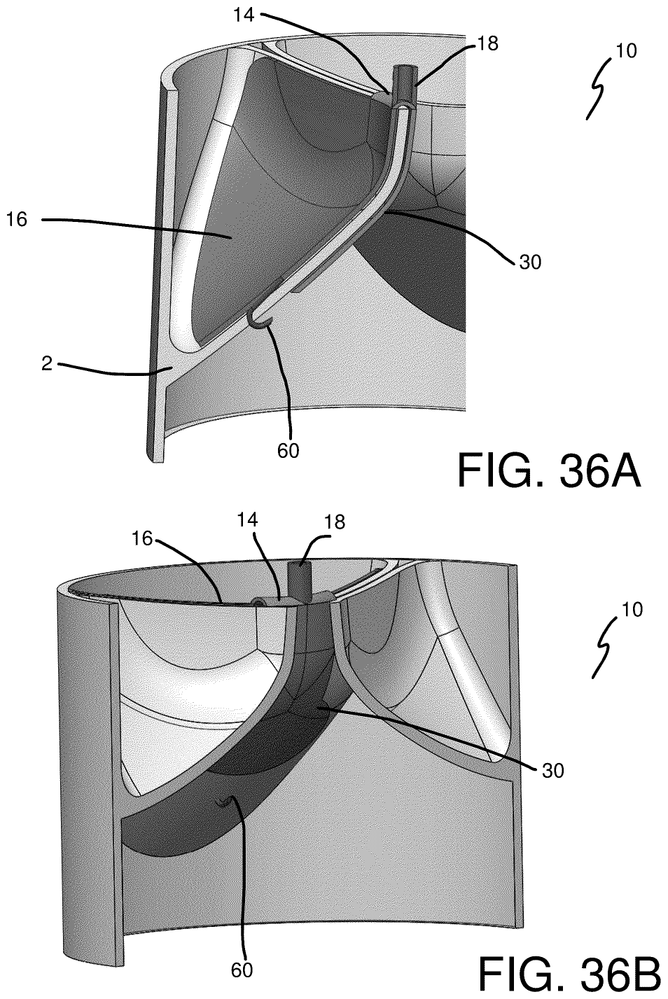

In certain implementations, one or both arms 12A, 12B and/or adhering surfaces 16A, 16B could include barbs, spikes, pins, or needles that are urged into the target leaflet 2 tissue when the clip 10 is in place. The barbs, spikes, pins, or needles may improve stability of the clip and attachment to the leaflet 2 tissue, shown variously in FIGS. 32A-36B.

Anchoring of the device on the leaflet could be via a passive mechanism related to the natural shape of the material used and ability to sandwich the leaflet tissue or via an active mechanism where one of the arms 12A, 12B acts as the anchor. In implementation with an active mechanism, the active forced mechanism may be a spring, loaded hinge, screw-in mechanism, or similar active mechanism, as would be understood

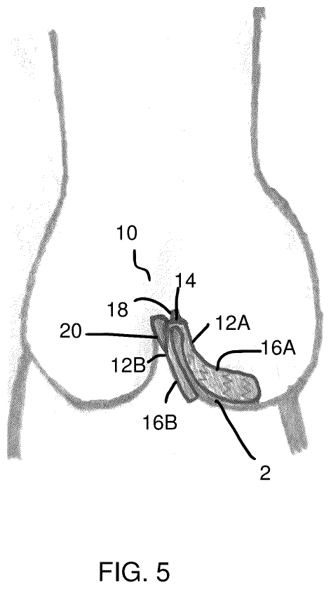

In various implementations, the device includes a spacer/central plug/extension 20, shown variously in FIGS. 5, 6, 11, 12, and 23-25. In these and other implementations, the spacer 20 is located at or near the hinge point 14, optionally approximately intermediate of the two arms 12A, 12B when the device 10 is symmetrical. The spacer 20 can be of various shapes and sizes to occlude a gap present between the various valve leaflets 2 when the valve is closed (diastole phase). The spacer 20 may be comprised of a polymeric material such as PTFE or other appropriate material, as would be appreciated by those of skill in the art. The spacer 20 will be discussed in further detail below.

In certain implementations the adhering surfaces 16A, 16B and other components of the clip 10 are foldable/compressible such as to fit within a catheter for delivery to the insertion site. In these implementations, the clip 10 and components thereof resume its uncompressed/unbent shaped once released into the body for positioning.

According to certain implementations, the clip 10 is delivered to the patient's valve via a minimally invasive procedure using a catheter and/or sheath, as would be appreciated. According to one specific implementation, the clip 10 is attached at a proximal attachment structure 18 or pin 18 to a delivery catheter, wire, or cable. In various implementations the attachment structure 18 is near the hinge point 14. The attachment structure/delivery feature/anchor is discussed further below in relation to FIGS. 19-22.

Further, in some implementations, shown in FIG. 2, strings 24A, 24B (or other similar elongate components) are threaded through rings 22A, 22B on each side of the clip 10, or along the body of the clip 10. In one example, a first string 24A is threaded through the ring(s) 22A of the first arm 12A and adhering surface 16A, and a second string 24B is threaded through the ring(s) 22B of the second arm 12B and adhering surface 16B. In this and other examples both strings 24A, 24B may extend proximally out of the patient such that the proximal ends of the strings 24A, 24B are accessible by a surgeon or other user use in delivery and positioning of the device 10.

In various implementations, the rings 22A, 22B face the opposite sides of the arms 12A, 12B and adhering surfaces 16A, 16B. The rings 22A, 22B may optionally be located on the sides of the arms 12A, 12B and about the circumference of adhering surfaces 16A, 16B or alternatively in the middle/along the body of the arms 12A, 12B and adhering surfaces 16A, 16B. In a further alternative, the rings 22A, 22B, and strings 24A, 24B may positioned at any appropriate point along the clip 10, as would be understood. As such, the clip 10 can be delivered to the target valve via the catheter such that the catheter is advanced in a minimally invasive manner through a blood vessel of the patient until the clip 10 is positioned as desired.

In use, any of the disclosed clip 10 implementations and related catheter and/or sheath implementations can be delivered to the target valve via cardiovascular access. More specifically, the clip 10 and delivery device (e.g., a catheter and/or guide wire) can be delivered via the femoral artery, radial artery, brachial artery, axillary artery, carotid artery, or any other similar artery or vessel. Further, in certain implementations, the delivery can be accomplished using visual guidance such as fluoroscopy and/or ultrasound technologies, as would be understood by those of skill in the art. Further, in certain implementations the delivery can be accomplished via other physical introduction technologies such as via a guide wire, as would be known and appreciated by those of skill in the art.

According to various implementations, any clip 10 implementation herein may optionally be recaptured after initial implantation and repositioned depending on the anatomical and functional needs before it is fully released by the delivery device.

FIGS. 13-38 show further implementations of a valve clip 10. Any of the features of the valve described in relation to FIGS. 2-12 may be applied to these implementations as would be understood. FIG. 13 shows a clip 10 having an adhering surface/leaflet portion 16 extending from a hinge point 14 and a short rim 30 extending from the hinge point 14 opposite the leaflet portion 16. In these and other implementations, the rim 30 extend along all or substantially all of the leaflet portion 16. In certain of these implementations, the rim 30 and leaflet potion 16 act together with the hinge point 14 to sandwich leaflet tissue. In these implementation, the leaflet portion 16 may be shaped and arranged to conform to the anatomy of the leaflet, optionally including a slight curvature and may optionally cover all or nearly all of the target leaflet when deployed.

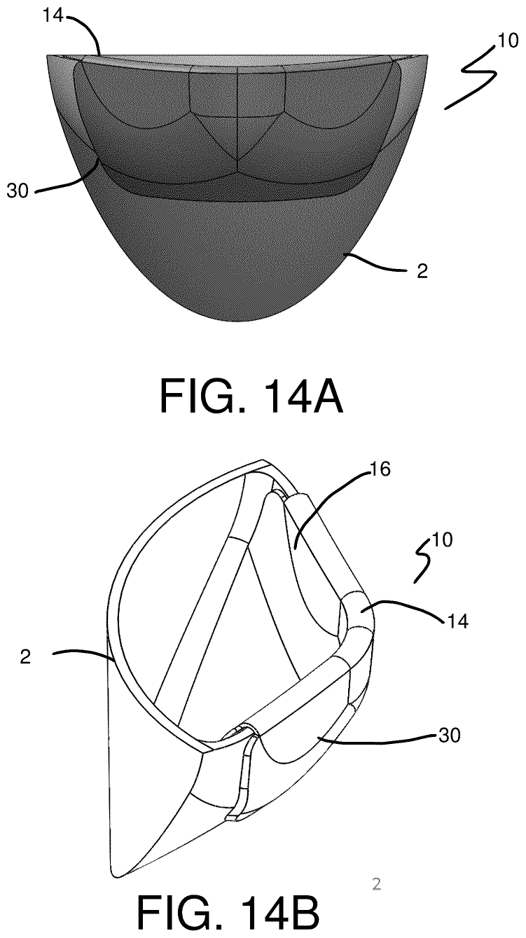

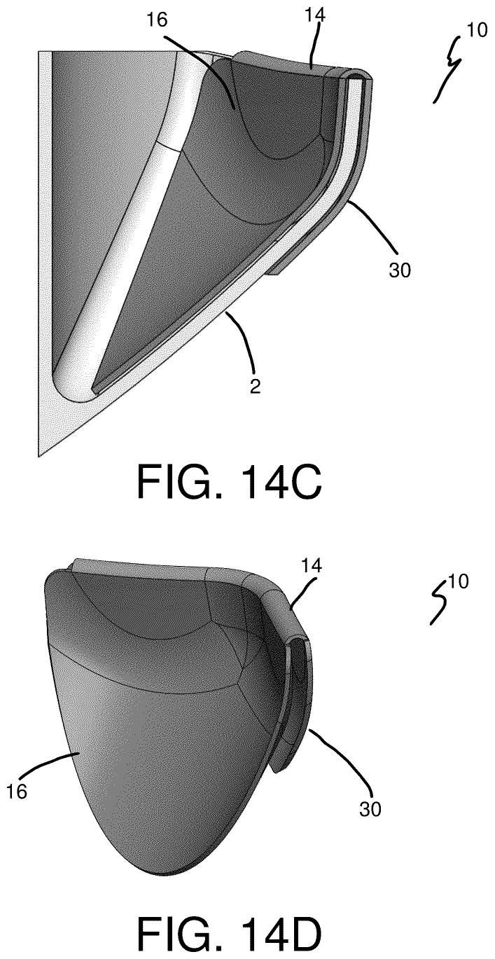

FIGS. 14A-D show a clip 10 having a leaflet portion 16 similar to that of FIG. 13 and including a rim 30 extending along all or nearly all of the leaflet portion 16. In these implementations, the rim 30 may be extended having a relatively longer piece to be substantially symmetrical with the leaflet portion 16 or otherwise extend along the leaflet portion. As can be seen, the extended rim may be of one unitary portion or a combination of pieces. In certain implementations, the leaflet portion 16 has a substantially triangular shape to match the anatomy of a tricuspid valve leaflet, but may differ based on the anatomy of the target valve. The rim 30 may be of a substantially rectangular or trapezoidal shape. In various implementations, the corners of the device 10 may have rounded or smoothed edges.

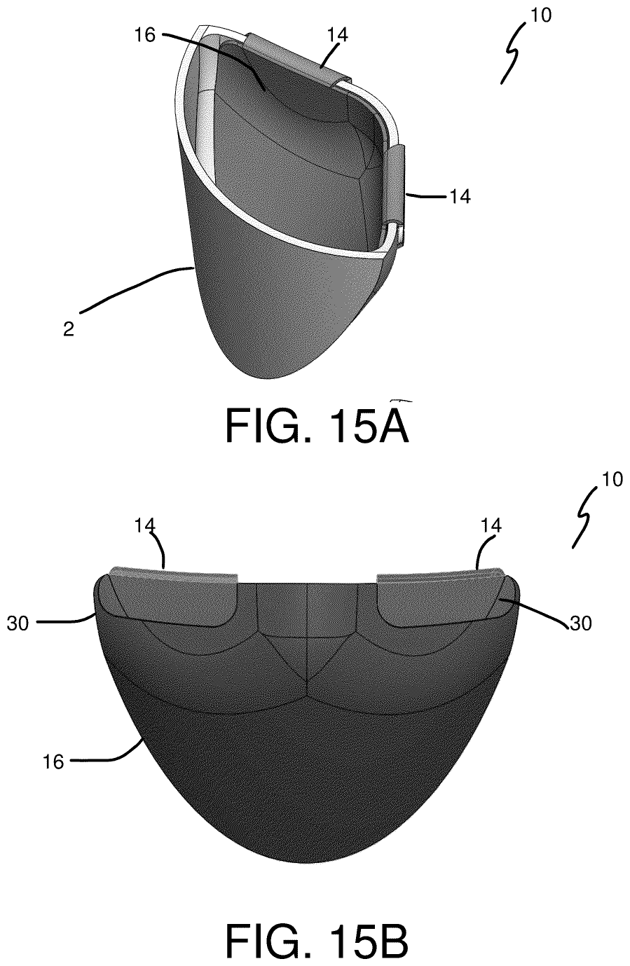

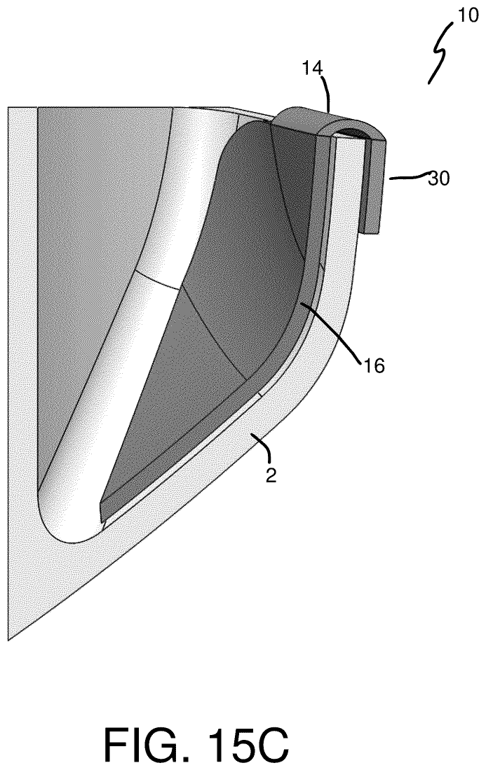

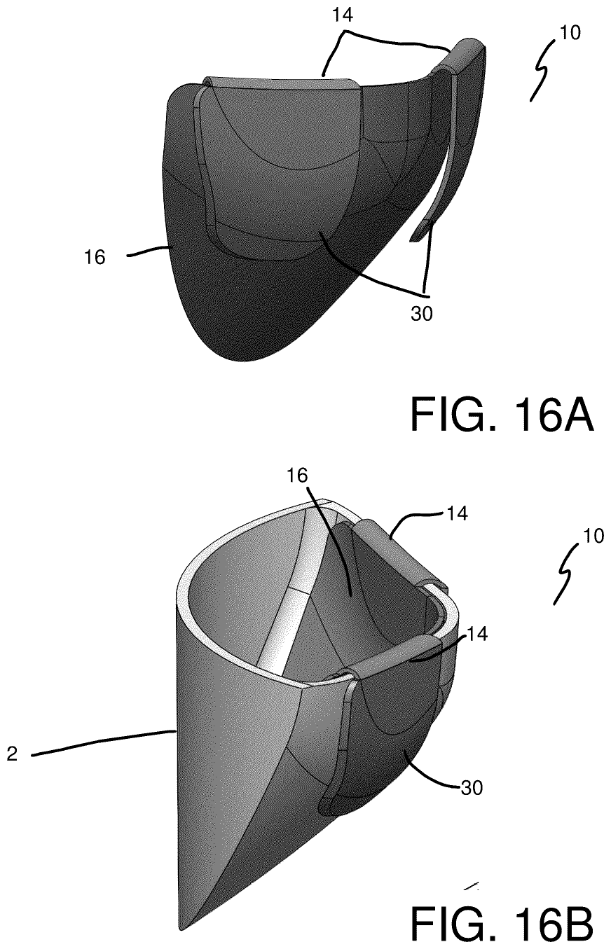

Continuing with FIGS. 15A-C and 16A-B, in various implementations, the device 10 may include a split rim 30. In these and other implementations, the rim 30 is bisected forming tow rim portions 30A, 30B on either side of the leaflet portion 16. The rim 30 may be a short rim extending only minimally on the second side of the leaflet opposite the leaflet portion (FIGS. 15A-C) or a long rim extending further along the second side of the leaflet opposite the leaflet portion (FIGS. 16A-B), a combination of short and long rims 30 are also possible. In these implementations, the center of the leaflet portion 16 is exposed. Optionally, when deployed in the valve, leaving the center exposed may aid in proper closure of the valve without a center plug, as discussed herein.

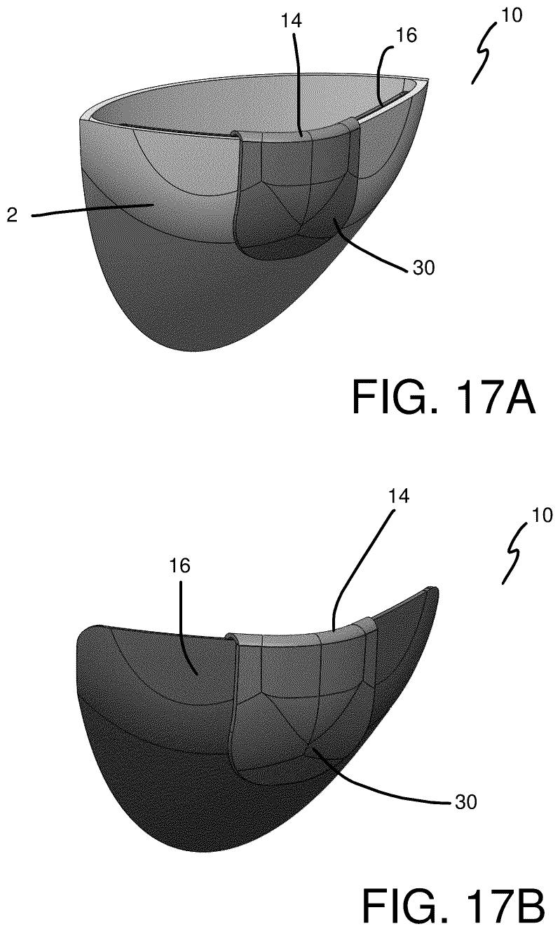

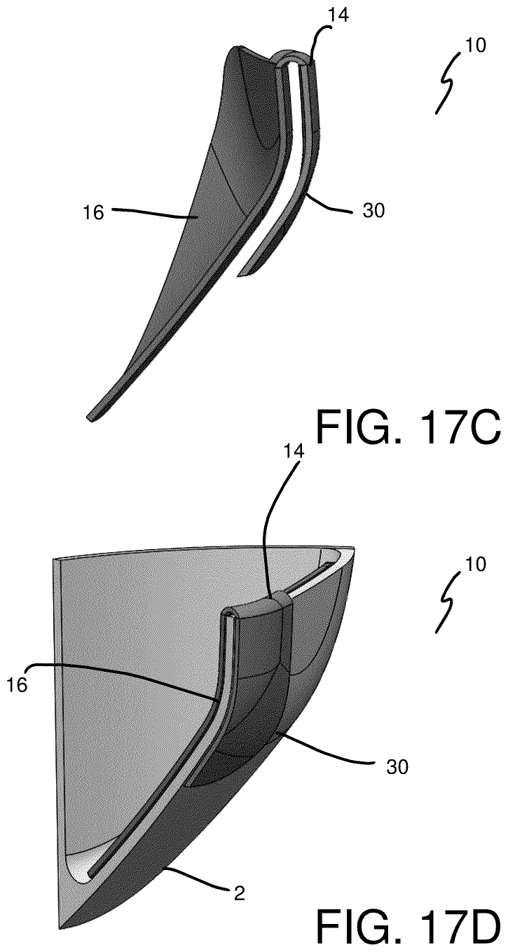

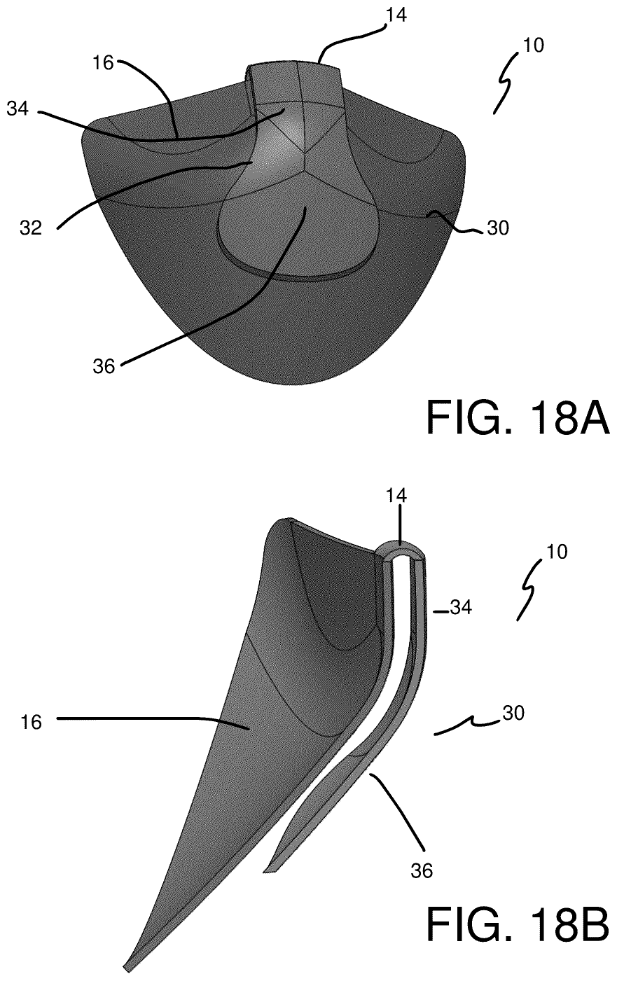

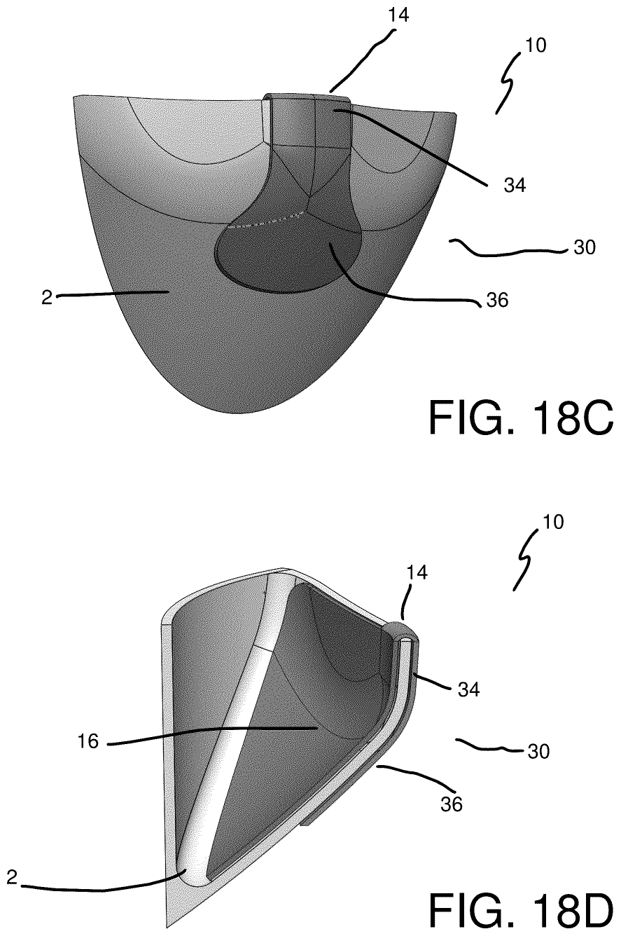

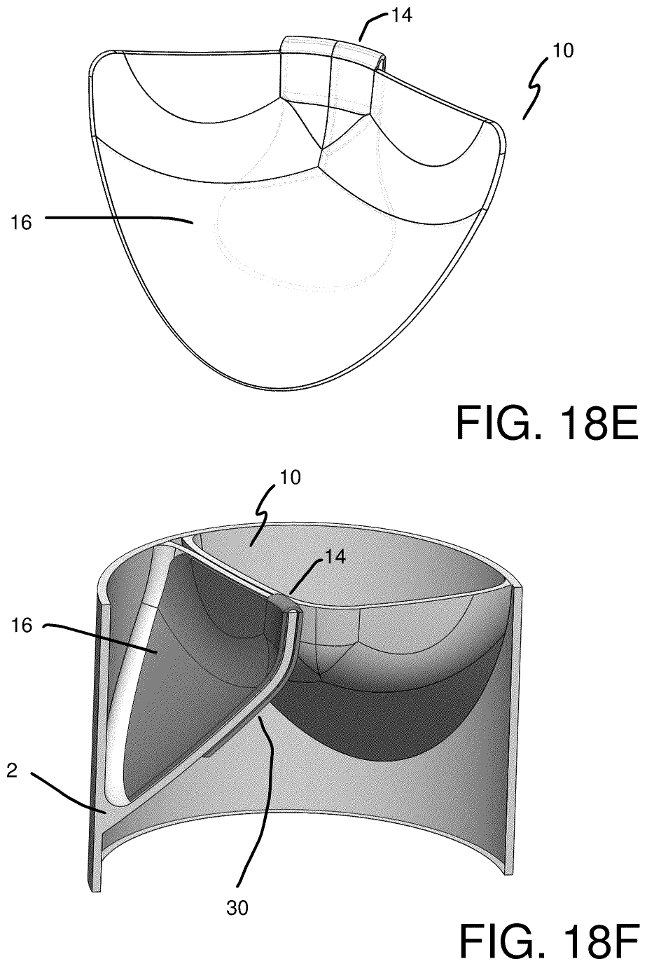

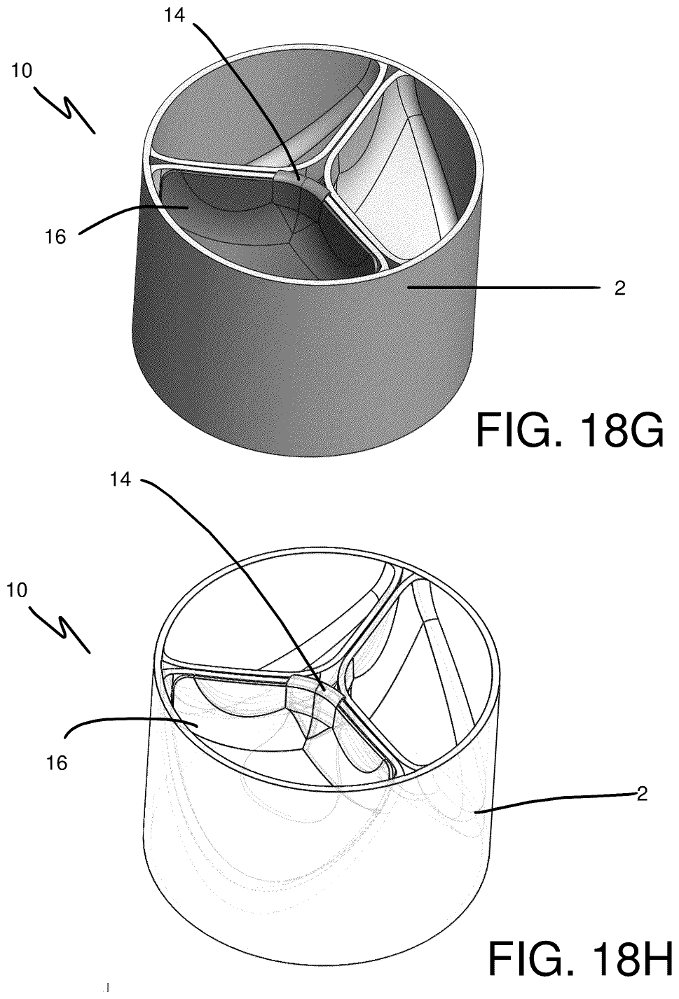

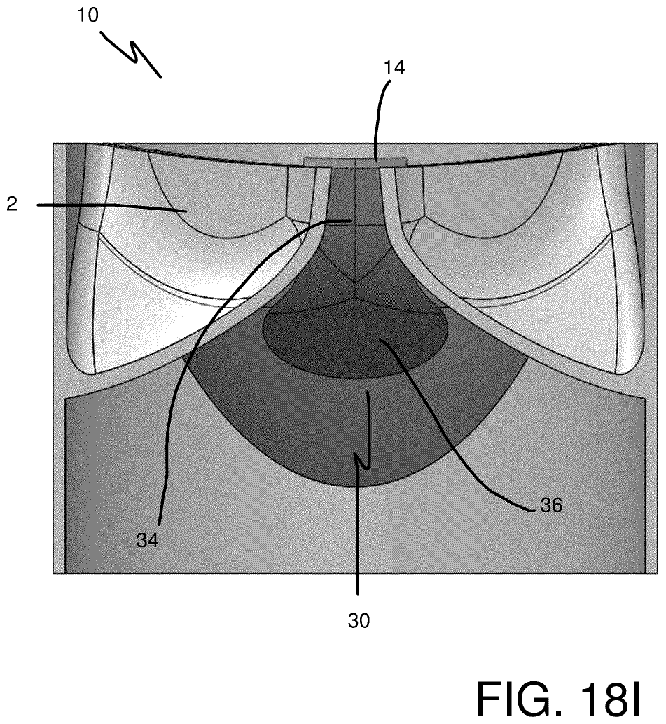

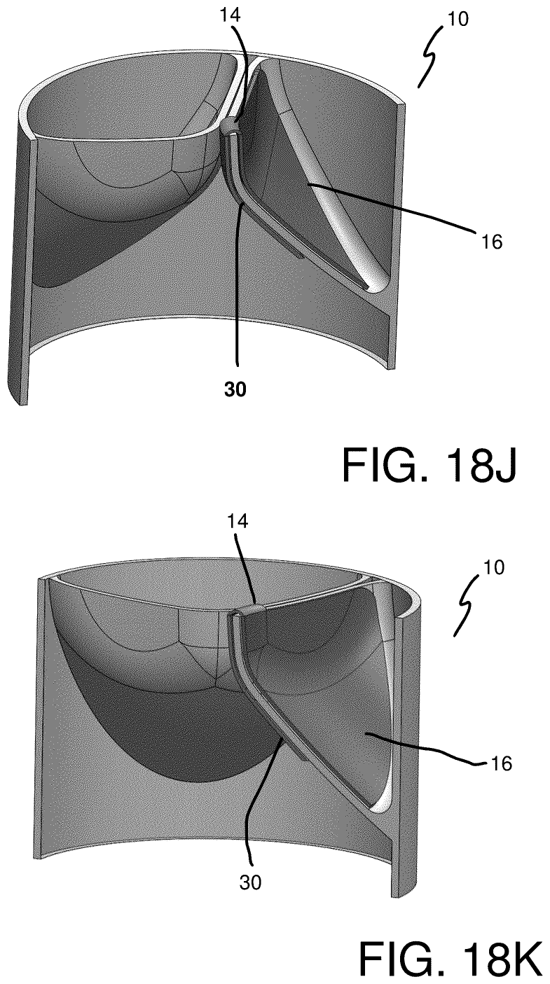

FIGS. 17A-18K show various further implementations of the device 10 including a rim 30 extending from the center of the leaflet portion 16 and hinge point and occupying a small (less than half) of the leaflet portion width. In these implementations, the rim 30 may have a short rim or long rim (shown in FIGS. 17A-D). In various implementations, the rim 30 may include a shaped extension portion 32, shown best in FIGS. 18A-K. The shaped extension portion 32 may include a first generally rectangular portion 34 and expanding into a rounded ovular or flared portion 36. Various alternative shapes are possible and would be appreciated. In these and other implementations, the extension portion 32 and flared portion 36 are shaped to provide additional support to the center of the leaflet. Various additional shapes and arrangement for the rim 30, extension portion 32, and leaflet portion 16 are possible and would be appreciated by those of skill in the art.

Turning now to FIGS. 19-23, in various implementations the device 10 includes a delivery feature/anchor 20. In these and other implementations, a loop, lock, or other similar anchor 20 is located on the device 10, optionally at the hinge point 14. The anchor 20 is shaped and engage with and attached to a delivery mechanism such as a catheter to deliver the device 10 to the target valve. Optionally, the anchor 20 may also include various feature to allow for manipulation of the device 10 for insertion and placement on the target leaflet.

As shown for example in FIG. 24-25, the device 10 may optionally include a central plug 20. The central plug 20 may extend from the hinge point 14, arm 12, or rim 30 at or near the center of the device 10. The central plug 20 being shaped to fill the center of the valve when the valve is closed to assist in proper valve sealing to prevent/minimize regurgitation. The center plug 20 may optionally be circular, semicircular, or any appropriate shape for the intended position.

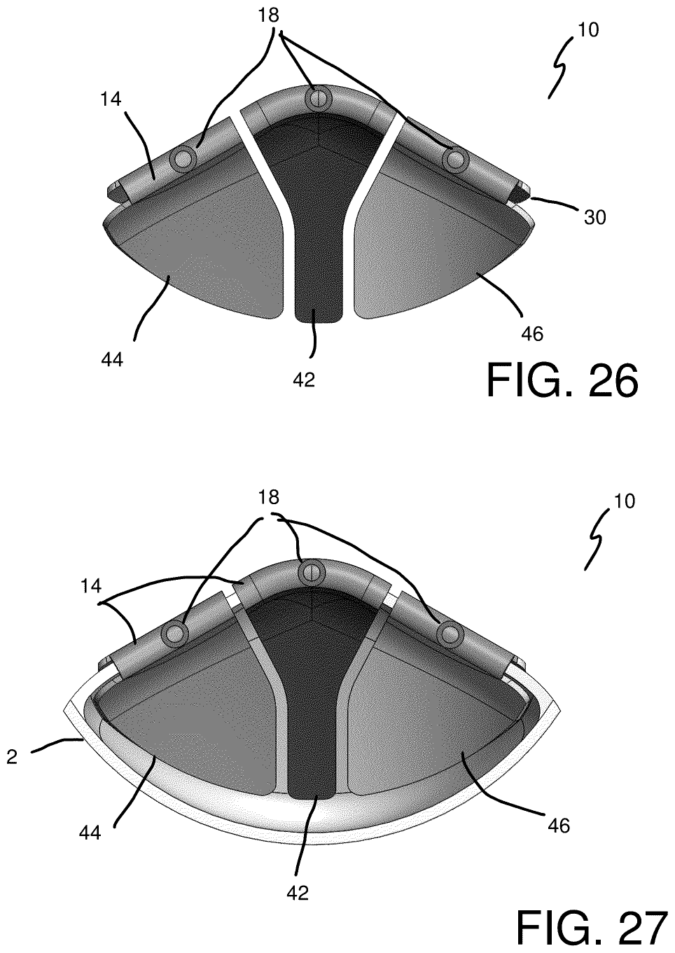

As can be in FIGS. 26-31, the clip 10 may be comprised of two, three, or more distinct components. In these and other implementations, the clip 10 includes a central component 42 and two side components 44, 46 placed near each other on the valve with certain spacing between the components. In these implementations, each component may include an anchor 20 for delivery and deployment. The split/divided clip 10 configuration may allow for additional flexibility in placement of the clip 10. Additionally, by having multiple components each is smaller than a unitary clip which may be useful during insertion and navigation to the target valve.

FIGS. 32A-36B show various view of the device 10 having hooks, barbs, spikes, pins, and/or needles that are urged into the target leaflet 2 tissue when the clip 10 is in place. The barbs, spikes, pins, or needles may improve stability of the clip and attachment to the leaflet 2 tissue. Any of the disclosed device 10 implementations disclosed herein may in include hooks 60 or similar connection pieces. Various numbers of hooks 60 may be present on the side of the leaflet portion 16 that contacts the leaflet. For example, one, two, three, or more hooks 60 may be present.

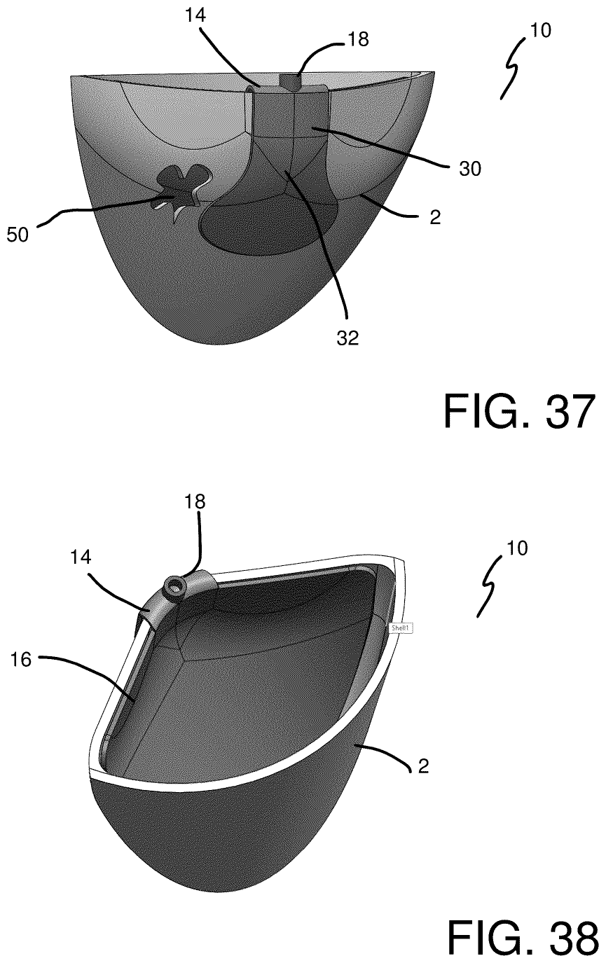

FIGS. 37 and 38, show an exemplary perforated valve leaflet 50. As can be seen, the leaflet portion 16 of the device 10 covers the perforated portion of the leaflet/valve wall, effectively sealing the leaflet and repairing the opening. The extension portion 32/rim 30 of the device, in this particular implementation, does not cover the perforated portion but instead covers the center portion of the leaflet 2 holding the device in place. As can be seen, the leaflet portion cover substantially all or all of the leaflet to ensure coverage of the perforated portion of the leaflet.

In various implementations, the device 10 may be used in conjunction with the devices, systems, and methods disclosed in U.S. patent application Ser. No. 63/428,639, filed Nov. 29, 2022.

Although the disclosure has been described with references to various embodiments, persons skilled in the art will recognize that changes may be made in form and detail without departing from the spirit and scope of this disclosure.

What is claimed is:

1. A valve clip, comprising:
(a) a first arm and a second arm attached at a hinge point; and
(b) an adhering surface at an end of the first arm,
wherein the hinge is configured for placement at a free edge of a valve leaflet and the first arm and second arm are on opposing sides of the valve leaflet.

2. The valve clip of claim 1, further comprising an anchor at the hinge point configured for attaching the valve clip to a delivery device.

3. The valve clip of claim 1, further comprising one of more hooks on a surface of the adhering surface.

4. The valve clip of claim 1, wherein the adhering surface comprises a membrane.

5. The valve clip of claim 1, wherein the first arm, second arm, and adhering surface are shaped from a frame comprising nitinol.

6. The valve clip of claim 1, wherein the adhering surface is shaped to cover substantially all of the valve leaflet.

7. The valve clip of claim 1, further comprising a center plug extending from the hinge point.

8. A method for repairing a valve comprising inserting via minimally invasive techniques valve clip of claim 1.

9. A clip for placement on a valve leaflet comprising:
(a) a leaflet portion configured for securement against a first side of the valve leaflet;
(b) a rear portion configured for securement against a second side of the valve leaflet; and
(c) a hinge point connecting the leaflet portion and the rear portion.

10. The clip of claim 9, wherein the rear portion comprises a rim extending along substantially all of the leaflet portion.

11. The clip of claim 9, wherein the rear portion comprises a split rim.

12. The clip of claim 9, wherein the rear portion comprises a flared extension extending from the center of the leaflet portion.

13. The clip of claim 9, wherein the leaflet portion is shaped to conform with leaflet anatomy and cover substantially all of the leaflet.

14. The clip of claim 9, further comprising an anchor point on the hinge point.

15. The clip of claim 9, further comprising a center plug extending from the hinge point.

16. The clip of claim 9, further comprising at least one hook extending from the leaflet portion configured for attachment to the leaflet.

17. The clip of claim 9, wherein the leaflet portion comprises a spring coil frame.

18. The clip of claim 9, wherein the leaflet portion comprises a membrane.

19. A method for repairing a valve leaflet, comprising inserting the device of claim 9 into a valve.

20. A leaflet clip for repairing a valve, comprising:
(a) a first arm having an adhering surface;
(b) a second arm;
(c) a hinge point connection the first arm and the second arm and forcing the first arm and second arm together;
(d) a delivery anchor disposed on the hinge point;
(e) a center plug extending from the hinge point; and
(f) at least one hook extending from the adhering surface.
